(19) United States
(12) Patent Application Publication
(10) Pub. No.: US 2013/0165526 A1
(43) Pub. Date: Jun. 27, 2013

(54) TOPICAL TREATMENT WITH DAPSONE IN G6PD-DEFICIENT PATIENTS

(71) Applicant: John S. Garret, Fort Collins, CO (US)

(72) Inventor: John S. Garret, Fort Collins, CO (US)

(73) Assignee: ALLERGAN, INC., Irvine, CA (US)

(21) Appl. No.: 13/528,488

(22) Filed: Jun. 20, 2012

Related U.S. Application Data
(63) Continuation of application No. 12/741,927, filed on Aug. 15, 2011, now abandoned.

(51) Int. Cl. A61K 9/00

(52) U.S. Cl. CPC A61K 9/0014
USPC 514/646

(57) ABSTRACT

The present invention provides a pharmaceutical carrier system comprising a dermatological composition that is a semi-solid aqueous gel, wherein dapsone is dissolved in the gel such that the dapsone has the capacity to cross the stratum corneum layer of the epidermis, and wherein the composition also contains dapsone in a microparticulate state that does not readily cross the stratum corneum of the epidermis. The present invention also provides methods of treating dermatological conditions in G6PD-deficient patients with the composition, while avoiding adverse hematologic effects.

### CROSS REFERENCE TO RELATED APPLICATIONS

This application is a continuation of U.S. application Ser. No. 12/741,927, filed Aug. 15, 2011, which is a national stage application under 35 U.S.C. §371 of PCT application PCT/US 2007/023468, filed on Nov. 7, 2007, all of which are incorporated herein by reference in their entirety.

### BACKGROUND OF THE INVENTION

Dapsone is a sulfone with both anti-inflammatory and antimicrobial properties. The oral formulation of the drug is used to treat leprosy, dermatitis herpetiformis, and malaria, using typical doses of 100 mg to 300 mg daily, but historically, it was also used to treat severe acne in doses ranging from 50 mg/day to 300 mg/week (Wolf et al., 2002; Ross 1961; Prendiville et al., 1988). Currently, use of oral dapsone is generally limited to more severe forms of skin disease, as its use may be associated with hematologic side effects, including hemolysis and hemolytic anemia that are dose-dependent and occur more frequently with increasing dose (Zhu and Stiller 2001; Jollow et al., 1995).

The mechanism of dapsone-related hemolysis and hemolytic anemia involves oxidative damage to red blood cells and is associated with the dapsone hydroxylamine metabolite (Prendiville et al., 1988). Red blood cells are somewhat protected against oxidative injury and lysis by glutathione reduction, a metabolic pathway that involves the glucose-6-phosphate dehydrogenase (G6PD) enzyme. Consequently, individuals who are G6PD-deficient are more sensitive to developing hemolytic anemia after exposure to hemolytic stressors such as infection, administration of a variety of drugs, including dapsone, or ingestion of fava beans (Beutler 1994). G6PD deficiency is most prevalent in individuals of African, Southeast Asian, and Middle Eastern heritage, and because the G6PD enzyme is encoded on the X chromosome, the deficiency is more common in males. In the United States, a recent study of military personnel reported the prevalence of G6PD deficiency to be 2.5% in men and 1.6% in women (Chinevere et al., 2006). Amongst racial groups, the prevalence was highest in African American men (12.2%), Asian men (4.3%), and African American women (4.1%), and lowest in Caucasian men and women (0.3% and zero, respectively). An early study that compared the effects of oral dapsone treatment in G6PD-deficient and non-deficient men found that there was a direct, linear relationship between oral dapsone dose and extent of red blood cell hemolysis in both the normal and deficient groups. The doses causing hemolysis in G6PD-deficient subjects were approximately half of the doses that caused hemolysis in subjects with normal G6PD levels (DeGowin et al., 1966).

What is needed is a method of treating dermatological conditions in patients including G6PD-deficient patients without the adverse hematologic effects associated with oral dapsone administration.

### SUMMARY OF THE INVENTION

The present invention provides methods to treat glucose-6-phosphate dehydrogenase-deficient patients with dapsone. In one embodiment, the treatment is directed to dermatological conditions and the treatment is provided by a topical dapsone composition. The composition may include dissolved dapsone and microparticulate dapsone. In certain embodiments, the dermatological condition to be treated is inflammatory acne, non-inflammatory acne or rosacea.

Second medical uses of the dapsone composition and methods of manufacture using the dapsone composition for treating dermatological conditions in a glucose-6-phosphate dehydrogenase-deficient patient are also contemplated by the present invention.

The present invention provides a pharmaceutical carrier system comprising a dermatological composition that is a semi-solid aqueous gel, wherein dapsone is dissolved in the gel such that the dapsone has the capacity to cross the stratum corneum layer of the epidermis and become available systemically, and wherein the composition also contains dapsone in a microparticulate state that does not readily cross the stratum corneum of the epidermis. The ratio of microparticulate to dissolved dapsone is adjustable, but is preferably five or less. Second medical uses of the dermatological composition and methods of manufacture of a medicament for treating dermatological conditions in a glucose-6-phosphate dehydrogenase-deficient patient using the dermatological composition are also contemplated by the present invention.

In some embodiments, the dermatological composition for use in methods of treating glucose-6-phosphate dehydrogenase-deficient patients includes a thickening agent; water; a high-boiling, nonionic organic solvent; a preservative; dapsone in a microparticulate and dissolved state; and a base solution. In one preferred embodiment, the composition includes about 0.5% to 4.0% carbomer; about 53.8% to 84.2% water; about 10% to 30% ethoxydiglycol; about 0.2% methylparaben; about 5% to 10% dapsone in a microparticulate and dissolved state; and about 0.1% to 2% sodium hydroxide solution. In some embodiments, the composition includes about 1% carbomer; about 81.8% water; about 10% ethoxydiglycol; about 0.2% methylparaben; about 5% dapsone in a microparticulate and dissolved state; and about 2% sodium hydroxide solution. In another preferred embodiment, the dermatological composition includes about 0.85% carbomer; about 66.95% water; about 25% diethylene glycol monoethyl ether; about 0.2% methylparaben; about 5% dapsone; and about 0.2% sodium hydroxide. Second medical uses of the dermatological composition and methods of manufacture of a medicament for treating dermatological conditions in a glucose-6-phosphate dehydrogenase-deficient patient using the dermatological composition are also contemplated by the present invention.

In certain embodiments, the invention provides a method to treat a dermatological condition in a glucose-6-phosphate dehydrogenase-deficient patient comprising applying topically a dermatological gel composition that includes a semisolid aqueous gel; dapsone dissolved in the gel, wherein the dapsone has the capacity to cross the stratum corneum layer of the epidermis and become available systemically; and a microparticulate dapsone dispersed in the gel, wherein the microparticulate dapsone does not cross the stratum corneum of the epidermis in its microparticulate state. The dermatological condition can include inflammatory acne, non-inflammatory acne and/or rosacea.

In embodiments where acne is treated, the acne can be non-inflammatory acne, inflammatory acne, or both. In some embodiments, the dermatological dapsone composition is a semisolid aqueous gel. In other embodiments, the dermatological dapsone composition is a cream or a lotion. In still other embodiments, the dapsone composition is a suspension, ointment, or spray. In each of these embodiments, the dapsone may exist as a microparticulate form, a dissolved form, or both.

In a preferred embodiment, the invention provides a method to treat a dermatological condition in a glucose-6-phosphate dehydrogenase-deficient patient by applying a dermatological composition to the condition, wherein the dermatological composition includes dapsone, wherein the method results in blood plasma levels of dapsone and N-acetyl dapsone below the levels associated with hemolysis. Second medical uses of the dermatological composition and methods of manufacture of a medicament for treating dermatological conditions in a glucose-6-phosphate dehydrogenase-deficient patient using the dermatological composition are also contemplated by the present invention.

In another preferred embodiment, the invention provides a method to treat a dermatological condition in a glucose-6-phosphate dehydrogenase-deficient patient by applying a dermatological composition to the condition, wherein the dermatological composition includes dapsone, and wherein the method results in blood plasma levels of dapsone and N-acetyl dapsone between about 0.5 μg/mL and 1.0 μg/mL. Second medical uses of the dermatological composition and methods of manufacture of a medicament for treating dermatological conditions in a glucose-6-phosphate dehydrogenase-deficient patient using the dermatological composition are also contemplated by the present invention.

In another preferred embodiment, the invention provides a method to treat a dermatological condition in a glucose-6-phosphate dehydrogenase-deficient patient by applying a dermatological composition to the condition, wherein the dermatological composition includes dapsone, and wherein the method results in blood plasma levels of dapsone and N-acetyl dapsone of about 1 μg/mL or less. Second medical uses of the dermatological composition and methods of manufacture of a medicament for treating dermatological conditions in a glucose-6-phosphate dehydrogenase-deficient patient using the dermatological composition are also contemplated by the present invention.

In another preferred embodiment, the invention provides a method to treat a dermatological condition in a glucose-6-phosphate dehydrogenase-deficient patient by applying a dermatological composition to the condition, wherein the dermatological composition includes dapsone, and wherein the method results in blood plasma levels of dapsone between 0 and about 37 ng/mL and blood plasma levels of N-acetyl dapsone between 0 and about 50 ng/mL. Second medical uses of the dermatological composition and methods of manufacture of a medicament for treating dermatological conditions in a glucose-6-phosphate dehydrogenase-deficient patient using the dermatological composition are also contemplated by the present invention.

In some embodiments, the method of treating a G6PD-deficient patient with dapsone results in blood plasma levels of dapsone less than about 37 ng/mL and blood plasma levels of N-acetyl dapsone less than about 50 ng/mL. In some preferred embodiments, the method of treatment does not induce hemolytic anemia. In some preferred embodiments, the methods do not induce adverse hematologic events. In still further embodiments, the method is performed for about 12 weeks.

The invention also provides a method to treat a dermatological condition in a glucose-6-phosphate dehydrogenase-deficient patient by topically applying a gel composition of dissolved dapsone and microparticulate dapsone, wherein the dissolved dapsone crosses the stratum corneum of the epidermis and is absorbed into the lower two-thirds of the pilosebaceous unit, and the microparticulate dapsone is primarily delivered into the upper third of the pilosebaceous unit, crossing the stratum corneum of the epidermis only minimally. Second medical uses of the dermatological composition and methods of manufacture of a medicament for treating dermatological conditions in a glucose-6-phosphate dehydrogenase-deficient patient using the gel composition are also contemplated by the present invention.

The use of a dermatological composition comprising about 0.85% carbomer; about 66.95% water; about 25% ethoxydiglycol; about 0.2% methylparaben; about 5% dapsone in a microparticulate and dissolved state; and about 0.2% sodium hydroxide solution, for the manufacture of a medicament for treating dermatological conditions in a glucose-6-phosphate dehydrogenase-deficient patient, is also contemplated by the invention.

In a preferred embodiment, the invention also provides a method to treat a dermatological condition in a patient by topically applying a dermatological composition including dapsone, wherein the dermatological composition is formulated to result in blood plasma levels of dapsone of less than 1 microgram per mL in the patient. In some embodiments, the patient is predisposed to hemolytic anemia. In some embodiments, the method results in blood plasma levels of dapsone less than about 37 ng/mL and blood plasma levels of N-acetyl dapsone less than about 50 ng/mL. In still further embodiments, the dermatological composition is a dermatological gel composition of a semisolid aqueous gel; dapsone dissolved in the gel, wherein the dapsone has the capacity to cross the stratum corneum layer of the epidermis and become available systemically; and a microparticulate dapsone dispersed in said gel, wherein the microparticulate dapsone does not cross the stratum corneum of the epidermis in its microparticulate state. Second medical uses of the dermatological composition and methods of manufacture of a medicament for treating dermatological conditions in patients using the gel composition are also contemplated by the present invention.

Methods for preparing the compositions of the present invention are also described.

### BRIEF DESCRIPTION OF THE FIGURES

FIG. 1. Study subject disposition. G6PD=glucose-6-phosphate dehydrogenase.

FIG. 2. Correlation analysis of the change in hemoglobin versus change in bilirubin at week 2 of dapsone gel treatment (r²=0.104; n=52). The mean bilirubin level was 0.58 mg/dL at baseline and 0.65 mg/dL at week 2. The mean change from baseline in bilirubin at week 2 (95% confidence limits) was +0.06 mg/dL (0 mg/dL, 0.12 mg/dL) (Patient data was collected in SI units and converted to conventional units for summary tables. To convert bilirubin mg/dL to SI units of μmol/L, multiply by 17.1). SI units=Systéme International units.

FIG. 3. Correlation analysis of the change in hemoglobin versus change in reticulocytes at week 2 of dapsone gel treatment (r²=0.043; n=52). The mean reticulocyte level was 1.30% at baseline and 1.51% at week 2. The mean change from baseline in reticulocyte level at week 2 (95% confidence limits) was +0.22% (0.11%, 0.32%).

FIG. 4. Correlation analysis of the change in hemoglobin versus change in haptoglobin at week 2 of dapsone gel treatment (r²=0.027; n=51). The mean haptoglobin level was 107.9 mg/dL at baseline and 109.1 mg/dL at week 2. The mean change from baseline in haptoglobin at week 2 (95% confidence limits) was −0.2 mg/dL (−5.3 mg/dL, 5.0 mg/dL) (Patient data was collected in SI units and converted to conventional units for summary tables. To convert haptoglobin mg/dL to SI units of g/L, multiply by 0.01). SI units=Systéme International units.

FIG. 5. Correlation analysis of the change in hemoglobin versus change in lactate dehydrogenase (LDH) at week 2 of dapsone gel treatment (r²<0.001; n=51). The mean LDH level was 175.0 IU/L at baseline and 171.3 IU/L at week 2. The mean change from baseline in LDH at week 2 (95% confidence limits) was −3.3 IU/L (−10.0 IU/L, 3.4 IU/L).

### DETAILED DESCRIPTION OF THE INVENTION

### Definitions

As used herein, “adverse event” means any adverse change in health or “side-effect” that occurs in a patient who is participating in a study while the patient is receiving treatment (dermatological composition or vehicle) or within a pre-specified period of time after their treatment has been completed.

As used herein, “cream” refers to an emulsified medicinal or cosmetic preparation; a semisolid emulsion of either the oil-in-water or the water-in-oil type, ordinarily intended for topical use.

As used herein, “dapsone” refers to the chemical compound dapsone having the chemical formula C₁₂H₁₂N₂O₂S as well as bis(4-aminophenyl)sulfone, 4′,4′-diaminodiphenyl sulfone and its hydrates, 4,4′-sulfonylbisbenzeneamine, 4,4′-sulfonyldianiline, diaphenylsulfone, dapsone analogs, and dapsone related compounds. “Dapsone analogs” refers to chemical compounds that have similar chemical structures and thus similar therapeutic potential to dapsone such as the substituted bis(4-aminophenyl)-sulfones. “Dapsone related compounds” refers to chemical compounds that have similar therapeutic potential, but are not as closely related by chemical structure to dapsone such as the substituted 2,4-diamino-5-benzylpyrimidines.

A “foam” refers to a mass of bubbles of air or other gas entrapped in a matrix of liquid or solid, especially an accumulation of fine, frothy bubbles formed in or on the surface of a liquid or solid, as from agitation or generated under pressure of a gas.

As used herein, the terms “G6PD-deficient” or “G6PD deficiency” refer to glucose-6-phosphate dehydrogenase (G6PD) levels that are below 7 U/g Hb, which is considered to be the lower limit of normal. As used herein, ≦2 U/g Hb is considered “severely” deficient.

As used herein, “gel” refers to a colloid in a more solid form than a solution; a jelly-like material formed by the coagulation of a colloidal liquid; many gels have a fibrous matrix and fluid filled interstices: gels are viscoelastic rather than simply viscous and can resist some mechanical stress without deformation.

As used herein, the term “microparticulate” refers to any solid form of an active agent, including dapsone, that is not dissolved in the dermatological composition. The microparticulate dapsone described herein may be in the form of flakes or crystals, and includes a precipitant that results from the addition of water and the solvent or mixed solvent system containing dapsone. The microparticulate dapsone may comprise a crystalline precipitant or an amorphous precipitant.

As used herein, “ointment” refers to a salve or unguent for application to the skin, specifically a semisolid medicinal preparation usually having a base of fatty or greasy material; an ointment has an oil base whereas a cream is water-soluble. See, The University of Newcastle Dept. of Medical Oncology On-Line Medical Dictionary (http://cancerweb.ncl.ac.uk/omd/) Dec. 19, 2003 and MedLine Plus Medical Dictionary (http://www.nlm.nih.gov/medlineplus/mplusdictionary.html) Dec. 19, 2003.

The term “topical” as used herein refers to the route of administration of a dermatological composition that involves direct application to the body part being treated, e.g., the skin. Examples of topical application include application to the skin of creams, lotions, gels, ointments or other semisolids to rub-on, solutions to spray, or liquids to be applied by an applicator. Rinse-off application with washes, cleansers, or shampoos are also examples of topical application. Typically, areas of the body suitable for application of the dermatological composition include the skin of the face, throat, neck, scalp, chest, back, ears, and other skin sites where dermatological conditions may occur.

As used herein, the term “treat”, “treatment”, or “treating” refers to the reduction in number and/or severity of symptoms, including individual skin lesions; prevention of the development of symptoms, including skin lesions; or global improvement in the appearance of symptoms, including skin lesions.

The invention described herein is directed to methods of treating dermatological disorders in G6PD-deficient patients through use of a topical dapsone formulation. Aczone™ gel, 5%, a topical formulation of dapsone, was developed to deliver therapeutic concentrations of dapsone to the skin. The United States Food and Drug Administration (US FDA) approved Aczone™ gel, 5%, for the treatment of acne vulgaris, but required certain language in the package insert due to the US FDA's concern that this drug carries a significant risk of serious hematological adverse effects, including hemolysis, in G6PD-deficient patients.

The US FDA required that the Aczone™ gel, 5%, label state that all patients should be screened for G6PD deficiency prior to initiation of Aczone™ treatment, with routine monitoring of complete blood counts and reticulocyte counts during treatment with Aczone™ in those patients identified as having a history of anemia and predisposition to increased hemolytic effect with dapsone (e.g., G6PD deficiency). While previous clinical studies did not demonstrate evidence of clinically significant anemia, an increased reticulocyte count and a decreased hemoglobin level were noted to be associated in a G6PD deficient patient treated with Aczone™ gel, 5% for acne vulgaris.

The methods and compositions of the invention described herein demonstrate the unexpected result that treatment of G6PD-deficient patients with the Aczone™ gel, 5%, formulation does not result in adverse hematological effects.

### Dapsone

Dapsone was first synthesized in 1908 and has been used medically as an antibiotic and an anti-inflammatory. Dapsone is a bis(4-aminophenyl)sulfone also known as 4′,4′-diaminodiphenyl sulfone, 4,4′-sulfonylbisbenzeneamine, 4,4′-sulfonyldianiline, and diaphenylsulfone. Dapsone has been used orally for the treatment of acne (Ross 1961) and been found to have a minimum inhibitory concentration with regard to P. acnes of about 1 microgram per milliliter (Godowski et al., 2000).

Dapsone analogs and related compounds have been described in U.S. Pat. Nos. 4,829,058 and 4,912,112 to Seydel et al. The '058 patent discloses substituted bis(4-aminophenyl)sulfones useful for inhibiting growth of bacteria, mycobacteria, and plasmodia. Some of these compounds were also tested against dapsone for toxicity and anti-inflammatory activity (Coleman et al., 1996a). In the '112 patent, substituted 2,4-diamino-5-benzyl pyrimidines having antimicrobial activity particularly against mycobacteria are described. Some of these compounds were also tested against dapsone for toxicity (Coleman et al., 1996b) and anti-inflammatory activity (Coleman et al., 1997). The teachings of these references in combination with subsequent publications showed that these analogs and related compounds have activity similar to dapsone and would be expected to have similar treatment efficacy.

### Topical Dapsone Compositions

The present invention comprises compositions for application to the skin of G6PD-deficient patients. The compositions comprise microparticulate dapsone precipitates in adjustable ratios of microparticulate to dissolved dapsone. The invention also comprises methods for preparation of the compositions, and methods for treatment of skin conditions in G6PD-deficient patients using the compositions. The advantages of the present invention are appreciated in the treatment of skin conditions or diseases by using topical dapsone, thus minimizing the hematologic effects associated with oral dapsone treatment. The present invention is particularly effective in the treatment of acne. Because of the nature of the microparticulate dapsone in the composition, microparticulate dapsone will be retained in or above the stratum corneum and will therefore serve as a reservoir or provide drug action in the supracorneum zone. The dissolved dapsone will pass through the stratum corneum.

Topical dapsone formulations have been described in U.S. Pat. No. 5,733,572 to Unger et al., and U.S. Pat. Nos. 6,056,954; 6,056,955; 6,254,866; 6,248,324; and 6,277,399 to Fischetti et al. A topical composition including dapsone for acne treatment has been described in U.S. Pat. Nos. 5,863,560, and 6,060,085 to Osborne which are herein incorporated by reference in their entirety.

Clinical studies have shown that dapsone gel, 5% (Aczone™; QLT USA, Inc. Fort Collins, Colo.) (dapsone gel), is effective in the treatment of acne vulgaris (Draelos et al., 2007) and results in <1% of the systemic exposure that is seen with typical oral dapsone treatment (Thiboutot et al., 2007).

Dapsone Topical Gel.

In a preferred embodiment, a dermatological condition in a G6PD-deficient patient is treated by topically applying a dermatological composition that is part of a novel pharmaceutical carrier system of a semisolid aqueous gel, wherein the composition exhibits an optimal balance between dissolved dapsone that is available to cross through the stratum corneum to become systemically available, and microparticulate dapsone that is retained in or above the stratum corneum to serve as a reservoir or to provide dapsone to the supracorneum zone. The microparticulate dapsone may comprise a crystalline precipitant or an amorphous precipitant.

Optimal balance is accomplished by having a semisolid gel carrier system in which microparticulate dapsone precipitates are formed in reproducible ratios with respect to the dissolved dapsone. For the composition to have a wide range of applicability, the microparticulate to dissolved dapsone ratio preferably should be no greater than five, at therapeutic levels of applied active dapsone.

A composition having a microparticulate to dissolved dapsone ratio of less than two may provide the greatest amount of pharmaceutical available for immediate partition out of the stratum corneum and into the viable epidermis. This should provide minimum reservoir capacity, but may not maintain sustained delivery or provide maximum activity in the supracorneum zone. A composition having a microparticulate to dissolved dapsone ratio of two or greater may have a reduced amount of drug available for immediate partition out of the stratum corneum and into the viable epidermis. This provides maximum reservoir capacity, and maintains sustained delivery, providing maximum activity in the supracorneum zone. In an example of a dermatological composition of this inventive method, the ratio for microparticulate dapsone to dissolved dapsone should be no greater than 50, preferably no greater than 10, and most preferably no greater than 5. Drug delivery from the microparticulate/dissolved dapsone formulation may be optimized to provide higher levels of drug to the supracorneum zone, while maintaining the level of drug partitioning out of the stratum corneum and into the viable epidermis, despite 10-fold increases in the amount of pharmaceutical applied to the skin.

Thickening agents include polymer thickeners. Polymer thickeners that may be used include those known to one skilled in the art, such as hydrophilic and hydroalcoholic gelling agents frequently used in the cosmetic and pharmaceutical industries. Preferably, the hydrophilic or hydroalcoholic gelling agent comprises “CARBOPOL®” (B. F. Goodrich, Cleveland, Ohio), “HYPAN®” (Kingston Technologies, Dayton, N.J.), “NATROSOL®” (Aqualon, Wilmington, Del.), “KLUCEL®” (Aqualon, Wilmington, Del.), or “STABILEZE®” (ISP Technologies, Wayne, N.J.). Preferably, the gelling agent comprises between about 0.2% to about 4% by weight of the composition. More particularly, the preferred compositional weight percent range for “CARBOPOL®” is between about 0.5% to about 2%, while the preferred weight percent range for “NATROSOL®” and “KLUCEL®” is between about 0.5% to about 4%. The preferred compositional weight percent range for both “HYPAN®” and “STABILEZE®” is between about 0.5% to about 4%.

“CARBOPOL®” is one of numerous cross-linked acrylic acid polymers that are given the general adopted name carbomer. These polymers dissolve in water and form a clear or slightly hazy gel upon neutralization with a caustic material such as sodium hydroxide, potassium hydroxide, triethanolamine, or other amine bases. “KLUCEL®” is a cellulose polymer that is dispersed in water and forms a uniform gel upon complete hydration. Other preferred gelling polymers include hydroxyethylcellulose, hydroxypropylcellulose, cellulose gum, MVA/MA copolymers, MVE/MA decadiene crosspolymer, PVM/MA copolymer, or a combination thereof.

Preservatives may also be used in this dermatological composition and preferably comprise about 0.05% to 0.5% by weight of the total composition. The use of preservatives assures that if the product is microbially contaminated, the formulation will prevent or diminish microorganism growth. Some preservatives useful in this invention include methylparaben, propylparaben, butylparaben, chloroxylenol, sodium benzoate, DMDM Hydantoin, 3-Iodo-2-Propylbutyl carbamate, potassium sorbate, chlorhexidine digluconate, or a combination thereof.

Titanium dioxide may be used as a sunscreen to serve as prophylaxis against photosensitization. Alternative sunscreens include methyl cinnamate. Moreover, BHA may be used as an antioxidant, as well as to protect ethoxydiglycol and/or dapsone from discoloration due to oxidation. An alternate antioxidant is BHT.

In one embodiment, the dermatological composition that is applied comprises a semi-solid or gel-like vehicle that may include a polymer thickener, water, preservatives, active surfactants or emulsifiers, antioxidants, sunscreens, and a solvent or mixed solvent system.

In a preferred embodiment, the dermatological composition includes a thickening agent; water; a high-boiling, nonionic organic solvent; a preservative; dapsone in a microparticulate and dissolved state; and a base solution. Ethoxydigylcol and 1-methyl-2-pyrollidone are preferred solvents for use in the topically applied dermatological composition. Sodium hydroxide is a preferred base for use in the topically applied dermatological composition. The solvent or mixed solvent system is important to the formation of the microparticulate to dissolved dapsone ratio. The formation of the microparticulate, however, should not interfere with the ability of the polymer thickener or preservative systems to perform their functions.

In one embodiment, the dermatological composition includes about 0.5% to 4.0% carbomer and about 0.5% to 10% dapsone that exists in both a dissolved state and a microparticulate state. In some embodiments, the dermatological composition comprises about 1% carbomer, about 80-90% water, about 10% ethoxydiglycol, about 0.2% methylparaben, and about 0.3% to 5.0% dapsone including both microparticulate dapsone and dissolved dapsone, and about 2% caustic base material. More particularly, the carbomer may include “CARBOPOL® 980” and the caustic base material may include sodium hydroxide solution.

In a preferred embodiment, the composition comprises dapsone and ethoxydiglycol, which allows for an optimized ratio of microparticulate drug to dissolved drug. This ratio determines the amount of drug delivered, compared to the amount of drug retained in or above the stratum corneum to function in the supracorneum domain. The system of dapsone and ethoxydiglycol may include purified water combined with “CARBOPOL®” gelling polymer, methylparaben, propylparaben, titanium dioxide, BHA, and a caustic material to neutralize the “CARBOPOL®”

In one embodiment, the dermatological composition that is applied comprises about 0.5% to 4.0% carbomer; about 73.8 to 82.3% water; about 10% ethoxydiglycol; about 0.2% methylparaben; about 5% to 10% dapsone in a microparticulate and dissolved state; and about 2% to sodium hydroxide solution. In another embodiment, the dermatological composition comprises about 1% carbomer; about 81.8% water; about 10% ethoxydiglycol; about 0.2% methylparaben; about 5% dapsone in a microparticulate and dissolved state; and about 2% sodium hydroxide solution. In one preferred embodiment, the composition comprises about 0.5% to 4.0% carbomer; about 53.8% to 84.2% water; about 10% to 30% ethoxydiglycol; about 0.2% methylparaben; about 5% to 10% dapsone in a microparticulate and dissolved state; and about 0.1% to 2% sodium hydroxide solution.

In a more preferred embodiment, the dermatological composition that is applied comprises about 0.85% carbomer, about 66.95% water, about 25% diethylene glycol monoethyl ether (i.e., ethoxydiglycol), about 0.2% methylparaben, about 5% dapsone, and about 0.2% sodium hydroxide solution.

Dapsone Topical Cream or Lotion.

In another embodiment, dapsone may be applied as a topical cream or lotion in which dapsone is dissolved or dispersed or both partially dissolved and partially dispersed. Topical creams or lotions may be either oil-in-water emulsions or water-in-oil emulsions. The oil phase may include but is not limited to fatty alcohols, acids, or esters such as cetyl palmitate, cetyl alcohol, stearyl alcohol, stearic acid, isopropyl stearate, glycerol stearate, mineral oil, white petrolatum, or other oils alone or in combination.

Emulsifiers that may be added to the composition include, but are not limited to, steareth 20, ceteth 20, sorbitan sesquioleate, sorbitan mono-oleate, propylene glycol stearate, dosium lauroyl sarcosinate, polysorbate 60, or combinations. Preservatives, antioxidants, fragrances, colorants, sunscreens, thickeners, and other additives required to achieve pharmaceutical or cosmetically acceptable or preferred product may also be included. However, topical creams and lotions are not limited to these components since one skilled in the art will be aware of additional components useful in the formulation of topical creams and lotions.

Dapsone Topical Solution or Suspension.

In another embodiment, dapsone may be applied as a solution or suspension. These are fluid solvent or mixed-solvent systems including, but not limited to, water, ethanol, propylene glycol, glycerol, polyethylene glycol, ethyl acetate, propylene carbonate, n-methylpyrolidone, triethanolamine, 1,4-butanediol, triacetin, diacetin, dimethyl isosorbide alone or in combination. Preservatives, antioxidants, fragrances, colorants, sunscreens, thickeners, suspending agents, enhancers, and other additives required to achieve pharmaceutically or cosmetically acceptable or preferred product may also be included. Again, topical solutions or suspensions are not limited to these components, since one skilled in the art will be aware of additional components useful in the formulation of topical solutions or suspensions.

Other Dapsone Topical Formulations.

Dapsone may also be applied using a pharmaceutical or cosmetic carrier form such as an ointment, roll-on or stick product, micro-emulsion, shake powder, an aerosolized spray or mousse, a pump spray or mousse, or bath additive. Examples of ointments include essentially non-aqueous mixtures of petrolatum, lanolin, polyethylene glycol, plant or animal oils, either hydrogenated or otherwise chemically modified. An ointment may also contain a solvent in which dapsone is either fully or partially dissolved. Additional pharmaceutical carriers will be known to those skilled in the art and this list should not be considered to be limiting.

### Method for Preparing the Dapsone Dermatological Composition

The present invention also provides methods for preparing the dermatological compositions described above. In a general form, the method for producing a dermatological gel composition having dissolved dapsone and microparticulate dapsone precipitates comprises the steps of completely dissolving dapsone in a solvent or solvent mixture; adding and adequately dispersing a polymeric thickener in water; and combining the dissolved dapsone with the dispersed polymeric thickener. Alternatively, water may be slowly added to the dissolved dapsone, followed by the addition of a polymeric thickener. Ethoxydigylcol and 1-methyl-2-pyrollidone are preferred solvents for use in the topically applied dermatological composition.

In one preferred embodiment, the method for preparing a topically applied dermatological composition having dissolved and microparticulate dapsone comprises the steps of forming a homogenous dispersion by stirring purified water vigorously enough to form a vortex and sifting gel polymer into the vortex formed in the water while continuing to stir; forming a pharmaceutical component by dissolving methyl paraben and/or propylparaben in ethoxydiglycol by mixing to form a solution, and mixing dapsone with the solution until the pharmaceutical is dissolved; mixing the pharmaceutical component with the homogenous dispersion to form a microparticulate dapsone dispersion; and adding a caustic material.

In a preferred embodiment, the method for preparing the topically applied dermatological composition having dissolved and microparticulate dapsone comprises the following steps: a polymer thickener component is prepared by charging 66.95 grams of purified water to a vessel suitable to contain 100 grams of finished semisolid product, and slowly sifting 0.85 g of “CARBOPOL® 980” into a vortex formed by rapidly stirring the purified water. When a homogeneous dispersion of “CARBOPOL® 980” and water is formed, stirring is reduced to minimize air entrapment. Next, an active pharmaceutical component is prepared by charging an appropriately sized container with 25 g of ethoxydiglycol, then 0.2 g of methylparaben are added to the ethoxydiglycol and mixed until all of the crystalline solid is dissolved. 5.0 g dapsone is added to the ethoxydiglycol and mixed until the drug is completely dissolved. The polymer thickener component is added to the pharmaceutical component with mixing, immediately resulting in the formation of crystalline microparticles. Once the dispersion is homogenous, 2.0 grams of a 10% w/w aqueous sodium hydroxide solution are added to neutralize the CARBOPOL® 980 and form the gel.

The order in which reagents are combined may be important, depending on the particular reagents necessary for the target mixture. For example, after a pharmaceutical such as dapsone is dissolved in a solvent such as ethoxydiglycol, water may be slowly added to the dapsone in the ethoxydiglycol solution, or the dapsone in ethoxydiglycol solution may be added to the water with mixing. Adding the dapsone in ethoxydiglycol solution to water may result in less polydispersity in the size of the microparticulates than adding water to the dapsone in ethoxydiglycol solutions. The carbomer is generally dispersed in the water component of the formulation, while the remaining ingredients will be dissolved or dispersed in whichever of the two components are best for dissolving or dispersing the ingredient. For example, it is suggested to dissolve methylparaben, propylparaben, and BHA in ethoxydiglycol. After the ethoxydiglycol component and water component are combined, neutralizer is added to formulate the gel.

The compositions of the present invention may further comprise other optional ingredients that may modify the physical, chemical, cosmetic or aesthetic characteristics of the compositions. The compositions may also further comprise optional inert ingredients. Many such optional ingredients are known for use in topical, including anti-acne compositions, and may also be used in the topical compositions herein, provided that such optional materials are compatible with the essential materials described herein, or do not otherwise unduly impair product performance.

The relative percentages for each of the reagents used in the present invention may vary depending upon the desired strength of the target formulation, gel viscosity, and the desired ratio of microparticulate to dissolved dapsone. Unless otherwise designated, all reagents listed above are commonly known by one of ordinary skill in the art and are commercially available from pharmaceutical or cosmetic excipient suppliers.

### Dermatological Conditions

The methods described herein treat dermatological conditions in G6PD-deficient patients by the topical application of a dermatologic composition comprising dapsone. In a preferred embodiment, acne conditions, e.g., inflammatory acne lesions and non-inflammatory acne lesions, are treated. In other embodiments, rosacea is treated.

Acne.

Acne is chronic pilosebaceous unit inflammation associated with the face and trunk, usually occurring in adolescence due to complex interactions of androgens and bacteria. For the adolescent, circulating androgen results in significantly increased sebum production. The sebaceous glands dramatically enlarge and excrete more sebum than the immature pilosebaceous canals can accommodate. The follicular canal contains keratinous material, i.e., dead skin cells, from the wall of the canal, sebum from the sebaceous glands, and bacteria, predominately Propionibacterium acnes. The P. acnes feed upon the sebum, converting triglycerides to fatty acids, and dramatically increase in number due to an increase in volume of the nutrition source. The increase in constricted immature ducts and bacterial waste products results in plugged follicles and subsequent typical acne inflammation.

When the follicular canal becomes blocked, a comedone is formed. The primary manifestation of non-inflammatory acne is the closed comedone, which are small, circumscribed, elevated lesions of the follicle that are often without a visible central plug. Closed comedones (whiteheads) are non-inflammatory acne lesions. Open comedones (blackheads) consist of small follicular lesions having a central black keratin plug as a result of oxidation of melanin pigment. Open comedones develop from closed comedones as the orifice dilates. The open comedone is not an inflammatory lesion unless traumatized, i.e. picked at, by the patient. Comedones, either open or closed, are non-inflammatory. While the comedone is the primary lesion of acne, comedones are not unique to acne since they may be seen in other conditions such as senile comedones or trophic skin resulting from x-ray therapy.

Closed comedones are potential precursors to large inflammatory lesions. The dead skin cells of the comedone are permeated with lipid and P. acnes, and as the follicle dilates from the expanding mass of keratin and lipid, inflammation develops along the follicular wall. This can lead to follicular wall rupture which extrudes the entire contents of the comedone into the dermis, generating a greater inflammatory response. Inflammatory lesions can be small papules with an encircling inflammatory region or, depending on the site and extent of the rupture, a pustule or large tender nodule may form. Papules, pustules and nodules are the three clinical descriptions for inflammatory acne.

As summarized by Strauss (J. S. Strauss. (1991). “Biology of the Sebaceous Gland and Pathophysiology of Acne Vulgaris,” Chapter 13 in Pathophysiology of Dermatologic Diseases, Second Edition. N. A. Sotor and H. Baden eds., McGraw-Hill, New York: pp. 195-210) there are four principles of acne therapy: 1) correct the pattern of altered keratinization within the follicle; 2) decrease sebaceous gland activity; 3) decrease the P. acnes population and/or decrease the generation of inflammatory substances by the bacterial population; and 4) produce non-inflammatory effects.

Topical retinoids such as tretinoin primarily function by correcting altered patterns of keratinization. Oral isotretinoin (13-cis retinoic acid) primarily functions by decreasing sebaceous gland activity. Known antibiotic therapies such as oral minocycline or topical clindamycin primarily function by reducing the numbers or activity of P. acnes.

Acne is one condition where a highly specialized topical drug delivery is needed. Ideally, a topical active agent would be primarily delivered into the pilosebaceous unit, with only minimal active crossing of the skin barrier. Intact stratum corneum lines the upper third of the pilosebaceous unit, and it is into this upper third of the hair follicle that the sebaceous duct secretes sebum. Thus, a need exists for an acne treatment that maximizes drug levels in the upper third of the pilosebaceous unit.

Additionally, when an active agent is used to treat acne, it is important to increase the level of drug that will cross the intact stratum corneum lining the upper third of the pilosebaceous unit. By definition, inflammation is the response of the viable epidermis to irritants and sensitizers. In order to reduce the amount of inflammation, the active pharmaceutical must penetrate past the stratum corneum and interfere with the cascade of inflammatory events. Ideally, delivery of an anti-inflammatory for acne requires that steady-state levels be sustained. The delivery system described herein provides dapsone above the stratum corneum and below the stratum corneum.

In one preferred embodiment of the invention, a method for treating acne in G6PD-deficient patients is employed by topically applying dapsone. Specifically, the invention includes a method for reducing the number of inflammatory acne lesions in G6PD-deficient patients by topically applying a dermatological composition comprising dapsone. The invention also includes a method for reducing the number of non-inflammatory acne lesions in G6PD-deficient patients by topically applying a dermatological composition comprising dapsone. Furthermore, in another embodiment, a method is provided for topically applying a dermatological composition comprising dapsone to prevent closed comedones (non-inflammatory acne) from becoming inflamed papules, pustules, or nodules in G6PD-deficient patients. However, if the follicular canal ruptures, dapsone would also help to reduce the resultant inflammation.

Rosacea.

Rosacea is estimated to affect over 45 million people worldwide. Early stages of rosacea are characterized by erythema (flushing and redness) on the central face and across the cheeks, nose, or forehead but can also less commonly affect the neck and chest. As rosacea progresses, erythema, telangiectasia (dilation of superficial blood vessels on the face), red domed papules (small bumps) and pustules, red gritty eyes, burning and stinging sensations, and in some advanced cases, a red lobulated nose (rhinophyma) develop. The disorder can co-exist with acne vulgaris and/or seborrheic dermatitis.

There are four identified rosacea subtypes (Wilkin et al., 2004) and patients may have more than one subtype present. First, erythematotelangiectatic rosacea is characterized by permanent redness (erythema) with a tendency to flush and blush easily. It is also common to have small blood vessels visible near the surface of the skin (telangiectasias) and possibly burning or itching sensations. Second, papulopustular rosacea is characterized by some permanent redness with red bumps (papules) with some pus filled (pustules) (which typically last 1-4 days). Third, phymatous rosacea is most commonly associated with rhinophyma, an enlargement of the nose. Symptoms include thickening skin, irregular surface nodularities, and enlargement. Phymatous rosacea can also affect the chin (gnatophyma), forehead (metophyma), cheeks, eyelids (blepharophyma), and ears (otophyma; Jansen and Plewig 1998). Small blood vessels visible near the surface of the skin (telangiectasias) may be present. Fourth, ocular rosacea is characterized by red, dry and irritated eyes and eyelids. Some other symptoms include foreign body sensations, itching and burning.

Rosacea may be triggered by episodes of skin flushing and blushing. Exposure to temperature extremes, strenuous exercise, heat from sunlight, severe sunburn, stress, anxiety, cold wind, moving to a warm or hot environment from a cold one, and foods and drinks containing alcohol, caffeine, histamines, spices, and antioxidants, can trigger flushing and blushing that contribute to the development of rosacea.

Other Dermatological Conditions.

In other embodiments of the invention, a topical dapsone formulation is used to treat G6PD-deficient patients suffering from impetigo, erythrasma, erysipelas, rosacea (perioral dermatitis, rhinophyma), furuncles, carbuncles, alopecia, panniculitis, psoriasis, dermatitis, cysts, bullous diseases (pemphigus vulgaris, bullous pemphigoid, and herpes gestationis), collagen vascular diseases (dermatomyositis, systemic lupus erythematosus, eosinophilic fasciitis, relapsing polychondritis, and vasculitis), sarcoidosis, Sweet's disease, lichen planus, hirsutism, toxic epidermal necrolysis, dermatitis herpetiformis, eczema, atopic dermatitis, seborrhoeic dermatitis (dandruff, cradle cap), diaper rash, urushiol-induced contact dermatitis, erythroderma, lichen simplex chronicus, prurigo nodularis, itch, pruritus ani, nummular dermatitis, dyshidrosis, pityriasis alba, parapsoriasis (pityriasis lichenoides et varioliformis acuta, pityriasis lichenoides chronica), pityriasis rosea, pityriasis rubra pilaris, urticaria (dermatographic urticaria, cholinergic urticaria), erythema (erythema nodosum, erythema multiforme, Stevens-Johnson syndrome, toxic epidermal necrolysis, erythema annulare centrifugum, erythema marginatum), sunburn, actinic keratosis, polymorphous light eruption, radiodermatitis, erythema ab igne, nail disease, onychogryposis, Beau's lines, yellow nail syndrome, follicular disorders, alopecia greata (alopecia universalis), androgenic alopecia, telogen effluvium, lichen planopilaris, trichorrhexis nodosa, hypertrichosis (hirsutism), epidermoid cysts, sebaceous cysts, pseudofolliculitis barbae, hidradenitis suppurativa, miliaria, anhidrosis, body odor, chromhidrosis, vitiligo, melasma, freckles, café au lait spots, lentigo/liver spots, seborrheic keratosis, acanthosis nigricans, callus, pyoderma gangrenosum, bedsores, keloids, granuloma annulare, necrobiosis lipoidica, granuloma faciale, morphea, calcinosis cutis, sclerodactyl), ainhum or livedoid vasculitis.

While the dermatological conditions described herein serve as examples of how therapeutic approaches can require dramatically different drug delivery profiles, all skin diseases are best treated by a particular drug delivery strategy tailored specifically to the pharmaceutical and the particular disease. Some diseases are best treated using pulsed or spiked delivery in which high levels of drug are delivered in a short period of time. This type of treatment saturates receptor sites and provides maximum microbial or viral replication inhibition, thus providing optimal therapy for certain diseases. Conversely, a cosmetic, topical, or transdermal product that provides steady state active pharmaceutical delivery while minimizing excipient delivery provides the preferred skin delivery profile for other diseases. Thus, the carrier system described herein, which can be adjusted to optimize the delivery profile for the pharmacology of the active drug and the nature of the disease state, advances the effectiveness of pharmaceutical products applied to the skin of G6PD-deficient patients.

The dapsone dermatological composition is typically applied to affected skin once or twice daily, but may be applied more frequently, depending on the severity of the condition. Hence, application may be as often as 3, or 4, or 5, or 6 times during a day, or even more. Typically, for most persons affected with acne, application once or twice during a day is sufficient.

The initial dosage, including frequency of the topical application and the length of the initial treatment period, can be determined depending on the specific type of dermatological condition, severity of the disease, and the response of the patient to the medication. The application should be repeated on a regular basis for at least 2 weeks in some embodiments, for at least 3 weeks in some embodiments, for at least 4 weeks in some embodiments, for at least 5 weeks in some embodiments, for at least 6 weeks in some embodiments, for at least 7 weeks in some embodiments, for at least 8 weeks in some embodiments, for at least 9 weeks in some embodiments, for at least 10 weeks in some embodiments, for at least 11 weeks in some embodiments, for at least 12 weeks in some embodiments, or longer in some embodiments. After elimination or reduction of the symptoms of the dermatological condition, application may be continued, or may be reduced to fewer times a day and/or fewer days a week to maintain the condition of the skin.

The dermatological compositions described herein can be sold as a kit wherein the composition is packaged in a container, such as a plastic container. Written instructions on how to use the dermatological composition in accordance with the present invention are included on or associated with the container, which provides instructions for treating dermatological conditions in G6PD-deficient patients.

The invention will be further described by reference to the following detailed, non-limiting examples.

### Example 1

### Hematologic Safety of Dapsone Topical Gel, 5%

### Methods

Study Design.

The study was a double-blind, randomized, vehicle-controlled, crossover, post-approval commitment study. Subjects were equally randomized into 1 of 2 sequences of treatment according to a computer-generated randomization scheme: dapsone gel followed by vehicle gel or vehicle gel followed by dapsone gel. The vehicle gel consisted of the same inactive ingredients as the dapsone gel. After washing with a standard, nonmedicated cleanser (Cetaphil, Galderma Laboratories, LP), subjects applied a thin film of the study treatment twice daily (once in the morning and once at night) to the entire face and, as required, to acne-affected areas of the neck, shoulders, upper chest, and upper back. Subjects applied each treatment for a period of 12 weeks, with a 2-week washout period between treatments and a 2-week follow-up period following the last treatment, for a total study duration of 28 weeks.

Study Treatment.

The method for preparing the topically applied dermatological composition having dissolved and microparticulate dapsone comprised the following steps: a polymer thickener component was prepared by charging 66.95 grams of purified water to a vessel suitable to contain 100 grams of finished semisolid product, and slowly sifting 0.85 g of “CARBOPOL® 980” into a vortex formed by rapidly stirring the purified water. When a homogeneous dispersion of “CARBOPOL® 980” and water was formed, stirring was reduced to minimize air entrapment. Next, an active pharmaceutical component was prepared by charging an appropriately sized container with 25 g of ethoxydiglycol, then 0.2 g of methylparaben were added to the ethoxydiglycol and mixed until all of the crystalline solid was dissolved. 5.0 g dapsone was added to the ethoxydiglycol and mixed until the drug was completely dissolved. The polymer thickener component was added to the pharmaceutical component with mixing, immediately resulting in the formation of crystalline microparticles. Once the dispersion was homogenous, 2.0 grams of a 10% w/w aqueous sodium hydroxide solution were added to neutralize the CARBOPOL® 980 and form the gel.

Subjects.

The subjects were age ≧12 years, had a diagnosis of G6PD deficiency (defined as having G6PD enzyme activity below the lower limit of normal; refer to Laboratory and Safety Assessments below), and had a diagnosis of acne vulgaris (defined as having at least 20 inflammatory and/or noninflammatory lesions, with at least 10 lesions located on the face). Subjects were excluded if they had severe cystic acne or acne conglobata, had received treatment with isotretinoin within 3 months of baseline, or were using other topical and/or systemic medications for acne at the time of study entry. Subjects were also excluded if they had a predisposition to anemia for other medical reasons, such as gastrointestinal bleeding or cancer.

Laboratory and Safety Assessments.

Adverse events were collected throughout the study with standard interviewing techniques at each study visit. Blood tests were scheduled for the baseline, 2-week, and 12-week time points of each treatment period to measure plasma dapsone and n-acetyl dapsone concentrations and evaluate clinical chemistry and hematology parameters. Lesion counts were assessed for efficacy at selected time points.

Blood samples from all subjects were tested for G6PD deficiency using a validated spectrophotometric assay performed with a commercially-available kit (Trinity Biotech PLC, Ireland). The laboratory's normal reference range for G6PD activity was 7.0 to 20.5 U/g Hb. Plasma dapsone and n-acetyl dapsone metabolite concentrations were measured by CANTEST BioPharma Services (Burnaby, British Columbia, Canada) using a validated liquid chromatography tandem mass spectrometry method. The lower limit of quantification for this assay was 0.30 ng/mL; levels below the lower limit of quantification were assigned a value of zero for the summary analyses. All clinical chemistry and hematology tests were analyzed centrally by Quintiles Laboratories (Smyrna, Ga., USA), which assigned a high or low flag to any values that were determined to be outside of the laboratory normal range.

Statistical Methods.

The intent-to-treat (ITT) population was defined as all randomized subjects, the safety population was defined as all subjects who applied dapsone gel or vehicle gel at least once, and the safety-evaluable population was defined as all subjects who applied at least 50% of the required treatment applications and had the Week 2 blood draw in the first treatment period. To assess the risk of hemolysis and hemolytic anemia, the following laboratory parameters were identified as important markers: hemoglobin, bilirubin, reticulocyte counts, haptoglobin, and lactate dehydrogenase (LDH). For each of these parameters, the values at each time point, changes from baseline at 2 and 12 weeks, and within-subject between-treatment differences in the values and changes from baseline were summarized with descriptive statistics (mean, standard deviation, median, minimum, and maximum). Two-sided 95% confidence intervals (CI) were also calculated for the changes from baseline and within-subject between-treatment differences in the change from baseline. In addition, the number and percentage of subjects with any of the following outcomes were determined for the 2-week and 12-week time point of each treatment period: a hemoglobin shift from normal or high to below normal or from low to normal or high; a hemoglobin reduction ≧1 g/dL; an increase in bilirubin above the upper limit of normal; an increase in reticulocyte count above the upper limit of normal; a reduction in haptoglobin below the lower limit of normal; and a ≧1 g/dL reduction in hemoglobin with concomitant increase in bilirubin, increase in reticulocytes, or reduction in haptoglobin. Unplanned correlation analyses between the changes in hemoglobin and changes in reticulocytes, bilirubin, haptoglobin, or LDH were performed with Pearson correlations. A preplanned subgroup analysis based on the degree of G6PD deficiency, which was defined as “severely deficient” (≦2 U/g Hb) and “deficient” (>2 U/g Hb up to the lower limit of normal at 7 U/g Hb) was performed for all variables related to the risk of hemolysis.

### Results

Subject Disposition and Demographic Characteristics.

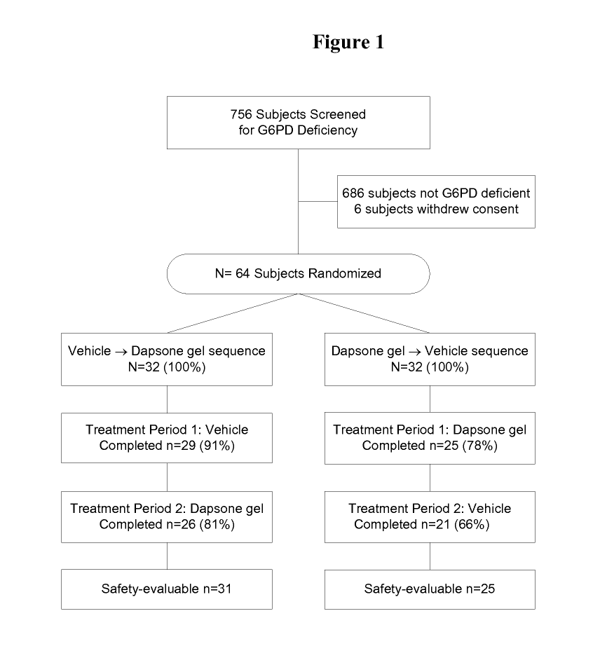

A total of 756 subjects were screened for G6PD deficiency; 64 subjects (8.5%) were identified as G6PD-deficient and consented to participation (FIG. 1). Of the 64 subjects in the intent-to-treat population, 63 comprise the safety population and 56 comprise the safety-evaluable population. Seventeen subjects did not complete the study, primarily for administrative reasons (loss to follow-up, voluntary withdrawal, treatment noncompliance, urticaria [not related to treatment], pre-existing anemia [protocol violation], pregnancy), but 1 of these subjects discontinued due to mild contact dermatitis.

Baseline demographics and characteristics were similar between treatment groups. The majority of subjects were African American (88%; 56/64) and the mean age of subjects was 28 years (Table 1). Six adolescent subjects (9%) were enrolled in the study (age <16 years). The mean of G6PD enzyme activity was 3.8 U/g Hb (range 0.7 to 6.9 U/g Hb). Fifteen subjects (23%) had severe G6PD-deficiency, defined as G6PD enzyme activity ≦2 U/g Hb, which represented the lower 30% of the below normal range; 14 of these subjects are included in the safety-evaluable population.

TABLE 1
Demographic and Baseline Characteristics (Intent-To-Treat)

| Characteristic | | |
|---|---|---|
| Total subjects, no. | 64 | |
| Age, mean ± SD (range), y | 28 ± 10 | (12-61) |
| Women, no. (%) | 35 | (55) |
| Ethnic group, no. (%) | | |
| African American | 56 | (88) |
| Asian | 4 | (6) |
| Hispanic | 1 | (2) |
| Other^{a} | 3 | (5) |
| Glucose-6-phosphate dehydrogenase | | |
| (G6PD) enzyme activity | | |
| Mean ± SD (range), U/g Hb | 3.8 ± 1.9 | (0.7-6.9) |
| Severely deficient,^{b} no. (%) | 15 | (23) |
| Deficient,^{b} no. (%) | 49 | (77) |
| Inflammatory lesion count, no. | | |
| Mean ± SD (range) | 16.2 ± 12.5 | (0-50) |
| Noninflammatory lesion count, no. | | |
| Mean ± SD (range) | 26.5 ± 25.4 | (0-139) |
| Total lesion count,^{c} no. | | |
| Mean ± SD (range) | 42.8 ± 27.8 | (10-162) |

SD = standard deviation
^{a}The subjects who identified their ethnic group as “other” were Middle Eastern, mixed race (White + Black), and Haitian.
^{b}Severely deficient is defined as ≦2 U/g Hb; deficient is defined as >2 U/g Hb up to the lower limit of normal at 7 U/g Hb
^{c}Total lesion count is the sum of inflammatory and noninflammatory lesion counts.

Lesion Counts.

Eficacy variables collected in this study were lesion counts (inflammatory, noninflammatory, and total). In all lesion categories, Aczone™-treated subjects experienced larger absolute reductions in lesions than vehicle-treated subjects after 12 weeks in the first treatment period. There was a higher percentage reduction in inflammatory lesion counts in Aczone™-treated subjects than vehicle-treated subjects (44% compared with 29%). Non-inflammatory lesion counts decreased by 5% in the Aczone™ group.

This was a cross-over design study that enrolled subjects with a baseline lesion count of at least 20 (mean total lesion count was 42.8 lesions). The primary purpose of this study was to evaluate safety, and therefore no statistical tests were planned for comparisons of the efficacy variables. The lesion counts were lower at the baseline of the second treatment period compared with the first, which indicates that the clinical effects of treatment last longer than the 2-week washout period and therefore, the evaluation of changes in lesion counts in treatment period 2 is confounded by the use of treatment during treatment period 1. Because of this, it is most relevant to evaluate changes in lesion counts over the first treatment period only.

After the first 12 weeks of the study, subjects treated with Aczone™ experienced a 44% drop in inflammatory lesion counts and 5% drop in non-inflammatory lesion counts. This pattern is consistent with the results from the pivotal phase 3 studies, in which Aczone™ demonstrated a larger effect on inflammatory lesions than noninflammatory lesions. Comparing Aczone™ and vehicle treatments in other lesion categories, it was observed that vehicle treatment in this study resulted in a better reduction in non-inflammatory lesion counts while the percentage reduction in total lesion count was similar between Aczone™ and vehicle. However, the absolute reduction in lesion counts was numerically better with Aczone™ treatment for all lesion categories. This variability in lesion counts is not unexpected given the small sample size of the study.

Plasma Dapsone and Metabolite Concentrations.

Dapsone and N-acetyl dapsone levels reached steady-state within 2 weeks of dapsone gel treatment and fell rapidly after the cessation of treatment. Mean plasma concentrations of dapsone were approximately 5 ng/mL at both 2 and 12 weeks of treatment, while mean plasma concentrations of n-acetyl dapsone were approximately 2.5 ng/mL at each time point (Table 2). In subjects who applied dapsone gel in the first treatment period, dapsone levels were largely undetectable by the baseline of the vehicle treatment period and completely undetectable by Week 2 of vehicle treatment (n=25, median dapsone concentration was 0, maximum concentration was 1.18 ng/mL at Week 2).

TABLE 2
Plasma Concentrations of Dapsone and N-Acetyl Dapsone for
Subjects Treated with Dapsone Gel, 5% (Safety Population)

| | | Dapsone | N-Acetyl Dapsone |
|---|---|---|---|
| | | (ng/mL) | (ng/mL) |
| 2 Weeks | n | 58 | 58 |
| | Mean ± SD | 5.626 ± 6.101 | 2.767 ± 6.750 |
| | Range | 0.00-36.85 | 0.00-48.70 |
| 12 Weeks | n | 51 | 51 |
| | Mean ± SD | 5.295 ± 6.660 | 2.514 ± 4.792 |
| | Range | 0.00-30.58 | 0.00-26.88 |

SD = Standard deviation

Hemolysis-Related Laboratory Results.

The primary hemolysis-related analysis was performed on the safety-evaluable data set (N=56). At 2 weeks of treatment, subjects treated with dapsone gel experienced a nominal mean decrease in hemoglobin from baseline of 0.32 g/dL (95% confidence limits −0.47 g/dL, −0.17 g/dL); no change from baseline was evident at 12 weeks of treatment (95% confidence limits −0.20 g/dL, 0.14 g/dL). In comparison, vehicle-treated subjects did not experience any changes in hemoglobin (Table 3). However, the within-subject between-treatment differences also show that subjects had hemoglobin values at 12 weeks of dapsone gel treatment that were similar to their level at 12 weeks of vehicle treatment.

The number of subjects who had a ≧1 g/dL hemoglobin decrease was similar between vehicle and dapsone gel treatment at Week 2 (4 subjects [7%] on vehicle compared with 6 subjects [11%] on dapsone gel). At Week 12, the number of subjects who had a ≧1 g/dL decrease was also similar between vehicle and dapsone gel treatment (4 subjects [7%] on vehicle with 2 subjects [4%] on dapsone gel). In addition, the range of hemoglobin changes from baseline was similar between vehicle and dapsone gel treatments, with changes in both positive and negative directions observed at each time point. The largest decrease in hemoglobin observed during the study occurred during vehicle treatment (1.7 g/dL decrease at Week 2).

After 2 weeks, hemoglobin shifts below normal were seen in 3 of 55 subjects (5%) during vehicle treatment and 6 of 52 subjects (12%) during dapsone gel treatment (Table 3). However, for both treatment groups, all of the low hemoglobin values remained close to the normal range and none of these subjects were diagnosed clinically with anemia. After 12 weeks, 4 of 50 subjects (8%) on vehicle and 3 of 49 subjects (6%) on dapsone gel experienced a hemoglobin shift to below the lower limit of normal. Three subjects experienced a shift in hemoglobin to below normal during both the vehicle and the dapsone gel treatment periods.

TABLE 3
Hemoglobin Values, Changes from Baseline, and Shifts
from Normal (Safety Evaluable Population)

| | Treatment Group | | Within-Subject |
|---|---|---|---|
| Visit | Vehicle gel | Dapsone gel, 5% | Difference^{a} |
| Pretreatment | n = 56 | n = 53 | n = 53 |
| Value, mean ± SD (g/dL) | 13.36 ± 1.25 | 13.44 ± 1.34 | −0.062 ± 0.593 |
| 2 Weeks | n = 55 | n = 53 | n = 52 |
| Value, mean ± SD (g/dL) | 13.34 ± 1.25 | 13.12 ± 1.36 | 0.238 ± 0.696 |
| Change from baseline, mean ± SD | 0.01 ± 0.64 | −0.32 ± 0.55 | 0.316 ± 0.957 |
| Range | −1.7 to 1.4 | −1.5 to 1.5 | −2.4 to 2.5 |
| 95% confidence interval^{b} | (−0.16, 0.18) | (−0.47, −0.17) | (0.047, 0.585) |
| ≧1 g/dL drop, no./n (%)^{c} | 4/56 (7%) | 6/56 (11%) | |
| Shift to below normal, no./n (%)^{d} | 3/55 (5%) | 6/52 (12%) | |
| 12 Weeks | n = 50 | n = 50 | n = 46 |
| Value (g/dL), mean ± SD | 13.37 ± 1.38 | 13.42 ± 1.24 | −0.024 ± 0.639 |
| Change from baseline, mean ± SD | 0.01 ± 0.64 | −0.03 ± 0.59 | 0.044 ± 0.913 |
| Range | −1.5 to 1.6 | −1.5 to 1.4 | −2.0 to 2.4 |
| 95% confidence interval^{b} | (−0.18, 0.19) | (−0.20, 0.14) | (−0.230, 0.319) |
| ≧1 g/dL drop, no./n (%)^{c} | 4/56 (7%) | 2/56 (4%) | |
| Shift to below normal, no./n (%)^{d} | 4/50 (8%) | 3/49 (6%) | |

SD = standard deviation
^{a}Difference is calculated as the vehicle value minus the dapsone gel value in the same subject
^{b}Confidence intervals are for the change from baseline (pretreatment value)
^{c}Denominators are the number of subjects at baseline
^{d}Based on observed data

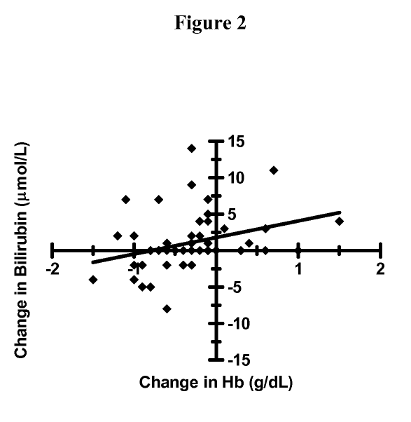

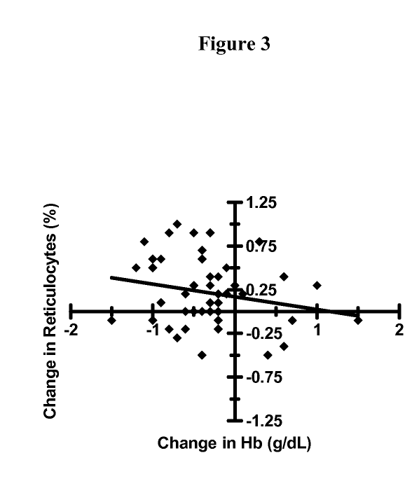

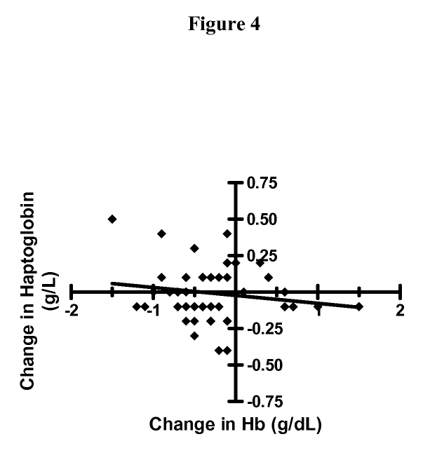

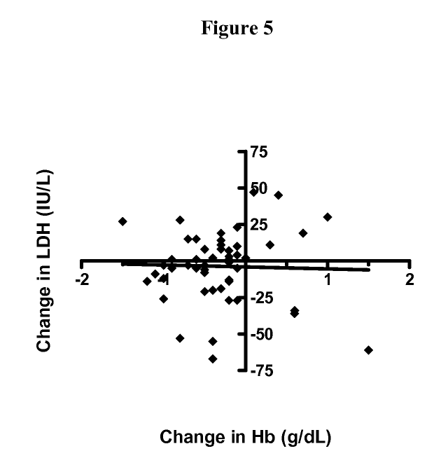

The changes in hemoglobin observed at week 2 were not correlated with plasma dapsone levels or grams per day of dapsone gel use (average use was 1 g/day). The changes in hemoglobin at week 2 were also not correlated with changes in bilirubin, reticulocytes, haptoglobin, or LDH (FIGS. 2 to 5). Correlation analyses showed that the regression lines between changes in bilirubin or haptoglobin and changes in hemoglobin slope in the opposite direction from those expected for hemolysis (ie, bilirubin would be expected to increase and haptoglobin would be expected to decrease in parallel with a decrease in hemoglobin) (FIGS. 2 and 4). No subjects experienced any concomitant changes of reticulocytes, haptoglobin, or LDH at 2 or 12 weeks of dapsone gel treatment. The various analyses of hemoglobin described above were similar between the safety evaluable and the safety populations of the study.

Two subjects experienced a change in hemoglobin of ≧1 g/dL with a concomitant increase of bilirubin to above the upper limit of normal (1 subject at 2 weeks of dapsone gel treatment and the other subject at 12 weeks). Neither subject experienced any other laboratory changes, particularly in the sensitive hemolysis marker haptoglobin, or clinical signs of hemolytic anemia. In addition, the subject with the changes at week 12 had similarly high bilirubin levels at week 2 of dapsone gel treatment and at week 12 of vehicle gel treatment, with no concomitant change in hemoglobin at these time points.

Changes in hemoglobin and other hemolysis parameters were also examined in various subgroups including G6PD enzyme activity, race, gender, and age. All of these subgroups demonstrated a similar pattern of changes in laboratory parameters as described for the safety-evaluable population. In particular, subjects who were severely G6PD-deficient (≦2 U/g Hb) did not appear to be at higher risk for changes in hemoglobin or other parameters (Table 4).

TABLE 4
Hemoglobin, Bilirubin, Reticulocyte, Haptoglobin, and Lactate
Dehydrogenase Levels by Severity of G6PD Enzyme Activity

| | | G6PD Enzyme Activity^{a} | | | |
|---|---|---|---|---|---|
| | | Severely Deficient^{b} (n = 14) | | Deficient^{b} (n = 42) | |
| Parameter | Visit | Vehicle gel | Dapsone gel, 5% | Vehicle gel | Dapsone gel, 5% |
| Hemoglobin (g/dL)^{c} | Pretreatment | 13.96 ± 0.80 | 13.97 ± 0.81 | 13.15 ± 1.32 | 13.25 ± 1.44 |
| | 2 Weeks | 13.91 ± 0.98 | 13.65 ± 0.79 | 13.14 ± 1.28 | 12.93 ± 1.48 |
| | 12 Weeks | 13.85 ± 0.85 | 13.86 ± 1.05 | 13.21 ± 1.50 | 13.24 ± 1.28 |
| Bilirubin (mg/dL)^{d} | Pretreatment | 0.7 ± 0.3 | 0.7 ± 0.2 | 0.5 ± 0.3 | 0.5 ± 0.3 |
| | 2 Weeks | 0.7 ± 0.3 | 0.8 ± 0.3 | 0.5 ± 0.2 | 0.6 ± 0.3 |
| | 12 Weeks | 0.8 ± 0.3 | 0.7 ± 0.4 | 0.6 ± 0.4 | 0.5 ± 0.3 |
| Reticulocytes (%) | Pretreatment | 1.38 ± 0.42 | 1.29 ± 0.45 | 1.33 ± 0.63 | 1.31 ± 0.47 |
| | 2 Weeks | 1.31 ± 0.44 | 1.59 ± 0.57 | 1.35 ± 0.55 | 1.48 ± 0.51 |
| | 12 Weeks | 1.45 ± 0.48 | 1.38 ± 0.53 | 1.39 ± 0.59 | 1.53 ± 0.61 |
| Haptoglobin (mg/dL)^{e} | Pretreatment | 93.57 ± 35.86 | 99.23 ± 30.13 | 118.10 ± 53.89 | 110.75 ± 48.17 |
| | 2 Weeks | 107.86 ± 29.66 | 102.14 ± 42.82 | 117.00 ± 48.42 | 111.54 ± 42.15 |
| | 12 Weeks | 95.38 ± 32.05 | 96.43 ± 30.54 | 117.03 ± 53.64 | 120.57 ± 50.11 |
| Lactate dehydrogenase | Pretreatment | 166.5 ± 34.5 | 162.0 ± 31.3 | 177.5 ± 38.9 | 179.6 ± 35.8 |
| (IU/L) | 2 Weeks | 165.1 ± 40.1 | 169.2 ± 32.1 | 179.0 ± 38.3 | 172.1 ± 31.6 |
| | 12 Weeks | 165.8 ± 33.4 | 174.1 ± 43.8 | 180.5 ± 36.8 | 177.1 ± 34.2 |

G6PD = glucose-6-phosphate dehydrogenase
^{a}Value ± standard deviation
^{b}Severely deficient is defined as ≦2 U/g Hb; deficient is defined as >2 U/g Hb up to the lower limit of normal at 7 U/g Hb
^{c}Patient data was collected in Systéme International (SI) units and converted to conventional units for summary tables. To convert g/dL to SI units of g/L, multiply by 10
^{d}To convert mg/dL to SI units of μmol/L, multiply by 17.1
^{e}To convert mg/dL to SI units of g/L, multiply by 0.01

One subject with pre-existing anemia at baseline and 2 subjects with histories of anemia were treated during the study. The subject with pre-existing anemia was treated with dapsone gel for 9 days before being withdrawn. However, despite being anemic at the start of dapsone gel treatment, she did not experience any worsening of hemoglobin or changes in other parameters after 9 days of treatment. The 2 subjects with histories of anemia completed the study. There were no changes in chemistry or hematology parameters indicative of dapsone-related hemolysis in either of these subjects during the study.

Adverse Events.

No adverse events were reported that were clinical signs or symptoms of hemolytic anemia. A total of 27 of 63 subjects (43%) in the full safety data set experienced an adverse event, regardless of relationship to treatment. Few adverse events were considered by the investigator to be related to dapsone gel treatment (17 events out of 44 events) and these occurred in only 8 of 63 subjects (13%): 7 during the dapsone gel treatment period and one during the vehicle treatment period. Four of these subjects reported local application site reactions of burning, dryness, pruritus, or contact dermatitis (all mild). The one event of contact dermatitis that led to discontinuation of study treatment was mild in intensity, did not require treatment, and resolved within 14 days of discontinuation. One subject reported a related adverse event of aggravated acne. Three subjects had related adverse events from a laboratory test result during treatment with dapsone gel, but none of these were indicative of hemolytic anemia: elevated bilirubin at 2 weeks and low hematocrit and low red blood cell count at 12 weeks in Subject 1; low haptoglobin at 2 weeks and RBC Burr cells, poikilocytosis, and elliptocytosis at 12 weeks in Subject 2; and low white blood cell count at week 12 in Subject 3. In Subject 1, the elevation in bilirubin occurred before the low hematocrit and RBC count and is therefore not believed to be indicative of hemolysis. In Subject 2, plasma dapsone levels were below the limit of quantification, even though treatment use could be verified by tube weights. Furthermore, the low haptoglobin was present at the baseline blood test as well as week 12, and no other changes in the other hematology parameters occurred, so the laboratory adverse events for this subject are likely not related to dapsone gel treatment. For Subject 3, no laboratory or clinical evidence of hemolysis or hemolytic anemia accompanied the low white blood cell count.

### Discussion

This study was designed specifically to evaluate the risk of hemolytic anemia with dapsone gel treatment in subjects with G6PD deficiency, which is a population at higher risk of drug-induced hematologic effects. The study employed a crossover design to evaluate both dapsone gel and vehicle treatments within the same subject. To evaluate hemolysis, subjects were monitored for changes in hemolysis-related laboratory parameters of hemoglobin, reticulocytes, haptoglobin, bilirubin, and LDH at 2 and 12 weeks of each treatment. Because drug-induced hemolytic anemia is a relatively acute phenomenon, the 2-week time point was determined to be the most relevant for observing any laboratory evidence of hemolysis or hemolytic anemia, while the 12-week time point would allow evaluation of any longer-term changes (Dern et al., 1954). Adverse events were also evaluated to determine if there were any clinical signs of hemolytic anemia.

An evaluation of the laboratory data showed a mean decrease in hemoglobin from baseline of 0.32 g/dL after 2 weeks of dapsone gel treatment, which was not seen at 12 weeks even as treatment continued. For several reasons, the nominal decrease in hemoglobin at Week 2 was considered to be clinically insignificant. First, there were no mean changes from baseline in other laboratory markers of hemolysis at either the 2-week or 12-week time point, nor any relationship between changes in hemoglobin and these parameters, including bilirubin, haptoglobin, reticulocytes, and LDH. These findings strongly argue against the presence of clinically relevant hemolysis. Second, no subjects experienced symptoms of or were diagnosed clinically with hemolytic anemia. No therapeutic interventions or modifications to study treatment were required as a consequence of a laboratory finding, even for subjects who experienced the largest decreases of hemoglobin (−1.7 g/dL and −1.5 g/dL for vehicle and dapsone gel treatment, respectively). Third, there was no consistent, clinically meaningful relationship between changes in hemoglobin and dapsone gel treatment. The range of hemoglobin changes and percentages of subjects with shifts below normal or large decreases of hemoglobin (≧1 g/dL) were similar between vehicle and dapsone gel treatments. Some subjects experienced changes in hemoglobin during both treatments, and some subjects had very low or unmeasurable plasma levels of dapsone.

This study also provides substantive data on a subgroup of 14 subjects whose G6PD levels were severely deficient, within the lower 30% of the G6PD-deficient range (≦2 U/g Hb). Results in this subgroup were consistent with the overall population and support that there is no difference in risk of hemolysis after dapsone gel treatment in G6PD-deficient subjects with the lowest enzyme activity. In addition, 1 subject with pre-existing anemia and 2 subjects with histories of anemia participated in the study. The subject with pre-existing anemia was withdrawn from the study after 9 days of treatment, but the other 2 subjects completed the full 28 weeks of the study. None of these 3 subjects experienced any changes in chemistry and hematology parameters indicative of dapsone-related hemolysis.

Because the G6PD enzyme is encoded on the X chromosome, the deficiency is generally more common in males, and the prevalence of G6PD deficiency in African-American men can be almost 3 times higher than that of African-American women (Chinevere et al., 2006). However, in this study, the ratio of males to females with G6PD deficiency was almost equal, with 55% (35/64) of eligible subjects being female. It could be speculated that because almost 60% of individuals who present at dermatologists' offices for skin concerns are female (Fleischer et al., 1994), and at least 41% of women will have acne at various times in their lives (Poli et al., 2001), investigators were able to identify a large number of female patients with both G6PD deficiency and acne. Subgroup analyses of all variables related to assessing the risk of hemolysis based on gender showed no differences between females and males, and the results for each subgroup were similar to the overall safety evaluable population.

Plasma dapsone and N-acetyl dapsone levels were measured pre-treatment and at the 2-week and 12-week time points of each treatment period to assess systemic exposure. To obtain a level of treatment exposure that was relevant to topical dapsone gel use in a real-world setting, subjects were instructed to apply treatment to the entire face twice-daily, and they could also apply treatment to other acne-affected areas of the neck, shoulders, upper chest, and/or upper back at their discretion. Dapsone and N-acetyl dapsone levels reached steady state within 2 weeks of treatment with dapsone gel, and fell rapidly after the cessation of treatment. Systemic exposure to dapsone after topical dapsone gel treatment was low, considering both the mean (approximately 5 ng/mL) and the maximum exposure in the study (approximately 37 ng/mL) (Table 2). The level of dapsone exposure observed in this study is substantially lower than the levels associated with oral dosing that would be expected to cause hematologic changes (DeGowin 1967). Hemolysis associated with oral dapsone use is a dose-dependent effect (Zhu and Stiller 2001; Jollow et al., 1995). We did not observe any correlation between plasma dapsone levels or grams of dapsone gel use and changes in hemoglobin, likely because systemic dapsone exposure was at the very low end of the dose-relationship observed with oral dapsone treatment. Pharmacokinetic modeling indicates that steady-state systemic dapsone levels after topical dapsone gel treatment would still be approximately 35-fold (Cₘₐₓ) to 63-fold (AUC) lower than the systemic levels of dapsone following a single 50 mg oral dose.

The results from this study demonstrate that there are no clinically significant effects on chemistry and hematology parameters or clinical signs of hemolytic anemia in G6PD-deficient subjects following treatment of acne vulgaris with dapsone gel. Because G6PD deficiency represents a highly sensitive marker for the hemolytic potential of drugs, this finding can be extrapolated to all acne patients with normal G6PD enzyme activity. Data from this study confirm that the safety profile for topical dapsone gel treatment is excellent, and support that the risk of hemolytic anemia during treatment with dapsone gel for acne vulgaris is remote for all patients, including those with G6PD deficiency.

All of the publications cited hereinabove are incorporated by reference herein. The invention has been described with reference to various specific embodiments and techniques. However, it should be understood that many variations and modifications may be made while remaining within the spirit and scope of the invention.

### BIBLIOGRAPHY

1. Beutler E. G6PD deficiency. Blood. 1994; 84:3613-3636.
2. Chinevere T D, Murray C K, Grant E Jr, Johnson G A, Duelm F, Hospenthal D R. Prevalence of glucose-6-phosphate dehydrogenase deficiency in U.S. Army personnel. Mil Med. 2006; 171:905-907.
3. Coleman et al., Environmental Toxicology and Pharmacology, 2:389-395(1996a).
4. Coleman et al., J. Pharm. Pharmacol., 48:945-950 (1996b)
5. Coleman et al., J. Pharm. Pharmacol., 49:53-57 (1997)
6. DeGowin R L, Eppes R B, Powell R D, Carson P E. The haemolytic effects of diaphenylsulfone (DDS) in normal subjects and in those with glucose-6-phosphate-dehydrogenase deficiency. Bull Wld Hlth Org. 1966; 35:165-179.
7. DeGowin R L. A review of therapeutic and hemolytic effects of dapsone. Arch Intern Med. 1967; 120:242-248.
8. Dern R J, Beutler E, Alving A S. The hemolytic effect of primaquine. II. The natural course of the hemolytic anemia and the mechanism of its self-limited character. J Lab Clin Med. 1954; 44(2):171-176.
9. Draelos Z D, Carter E, Maloney J M, et al. Two randomized studies demonstrate the efficacy and safety of dapsone gel, 5% for the treatment of acne vulgaris. J Am Acad Dermatol. 2007; 56:439-447.
10. Fleischer A B, Feldman S R, Bradham D D. Office-based physician services provided by dermatologists in the United States in 1990. J Invest Dermatol. 1994; 102:93-97.
11. Godowski K L et al., J. Invest. Dermatol. 114:862 (2000)
12. Jansen T, Plewig G. Clinical and histological variants of rhinophyma, including nonsurgical treatment modalities. Facial Plast Surg 14 (4): 241-53 (1998). PMID 11816064.
13. Jollow D J, Bradshaw T P, McMillan D C. Dapsone-induced hemolytic anemia. Drug Metab Review. 1995; 27:107-124.
14. Poli F, Dreno B, Verschoore M. An epidemiological study of acne in female adults: results of a survey conducted in France. J Eur Acad Dermatol Venereol. 2001; 15:541-545.
15. Prendiville J S, Logan R A, Russell-Jones R. A comparison of dapsone with 13-cis retinoic acid in the treatment of nodular cystic acne. Clin Exp Dermatol. 1988; 13:67-71.
16. Ross C M. The treatment of acne vulgaris with dapsone. Br J Dermatol. 1961; 73:367-370.
17. Thiboutot D, Willmer J, Sharata H, Halder R, Garrett S. Pharmacokinetics of dapsone gel 5% for the treatment of acne vulgaris. Clin Pharmacokinet. 2007; 46:697-712.
18. Wilkin J, Dahl M, Detmar M, Drake L, Liang M H, Odom R, Powell F. Standard grading system for rosacea: report of the National Rosacea Society Expert Committee on the classification and staging of rosacea. J Am Acad Dermatol 50 (6): 907-12 (2004). PMID 15153893.
19. Wolf R, Matz H, Orion E, Orion E, Tuzun B, Tuzun Y. Dapsone. Dermatol Online J [serial online]. 2002; 8:2. Available at: http://dermatology.cdlib.org/DOJvol8num1/reviews/dapsone/wolf.html. Accessed Sep. 21, 2007.
20. Zhu Y I, Stiller M J. Dapsone and sulfones in dermatology: overview and update. J Am Acad Dermatol. 2001; 45:420-434.

What is claimed is:

1. A method to treat a dermatological condition in a glucose-6-phosphate dehydrogenase-deficient patient comprising applying a dermatological composition to said condition, wherein said dermatological composition comprises dapsone.

2. The method of claim 1, wherein the dermatological composition comprises dissolved dapsone and microparticulate dapsone.

3. The method of claim 1, wherein the dermatological condition is selected from the group consisting of inflammatory acne, non-inflammatory acne and rosacea.

4. A method to treat a dermatological condition in a glucose-6-phosphate dehydrogenase-deficient patient comprising applying topically a dermatological gel composition including microparticulate pharmaceutical and dissolved pharmaceutical, which comprises:
a thickening agent;
water;
a high-boiling, nonionic organic solvent;
a preservative;
dapsone in a microparticulate and dissolved state;
and a base solution.

5. The method of claim 4, wherein the ratio of microparticulate to dissolved dapsone is no greater than 5.

6. The method of claim 4, wherein the dermatological condition is selected from the group consisting of inflammatory acne, non-inflammatory acne and rosacea.

7. A method to treat a dermatological condition in a glucose-6-phosphate dehydrogenase-deficient patient comprising applying topically a dermatological gel composition comprising:
a semisolid aqueous gel;
dapsone dissolved in said gel, wherein said dapsone has the capacity to cross the stratum corneum layer of the epidermis and become available systemically; and
a microparticulate dapsone dispersed in said gel, wherein said microparticulate dapsone does not cross the stratum corneum of the epidermis in its microparticulate state.

8. The method of claim 7, wherein the dermatological condition is selected from the group consisting of inflammatory acne, non-inflammatory acne and rosacea.

9. A method to treat acne in a glucose-6-phosphate dehydrogenase-deficient patient comprising applying topically a dermatological composition comprising dapsone.

10. The method of claim 9, wherein the acne is non-inflammatory acne.

11. The method of claim 9, wherein the acne is inflammatory acne.

12. The method of claim 9, wherein the dermatological composition is selected from the group consisting of a semisolid aqueous gel, a cream, a lotion, a suspension, an ointment and a spray.

13. The method of claim 9, wherein the dermatological composition comprises dissolved dapsone and microparticulate dapsone.

14. The method of claim 1, wherein the method results in blood plasma levels of dapsone less than about 37 ng/mL and blood plasma levels of N-acetyl dapsone less than about 50 ng/mL.

15. The method of claim 1, wherein the method does not induce hemolytic anemia.

16. The method of claim 1, wherein the method does not induce adverse hematologic events.

17. The method of claim 1, wherein the method is performed for about 12 weeks.
